(19) United States
(12) Patent Application Publication
(10) Pub. No.: US 2015/0099898 A1
(43) Pub. Date: Apr. 9, 2015

(54) PROCESS FOR REMOVING AMIDE IMPURITIES IN AROMATIC CARBOXYLIC ACIDS

(71) Applicant: UOP LLC, Des Plaines, IL (US)

(72) Inventors: Avram M. Buchbinder, Chicago, IL (US); Alakananda Bhattacharyya, Glen Ellyn, IL (US); Tatsamai T. Jarusiripipat, Chicago, IL (US); Raymond Shih, Elgin, IL (US); Joel T. Walenga, Lake Zurich, IL (US); Nina Kuznetsova, Novosibirsk (RU)

(73) Assignee: UOP LLC, Des Plaines, IL (US)

(21) Appl. No.: 14/048,582

(22) Filed: Oct. 8, 2013

(51) Int. Cl. C07C 51/16

(52) U.S. Cl. CPC C07C 51/16
USPC 562/410

(57) ABSTRACT

A process for oxidizing alkyl aromatic compounds is described. The alkyl aromatic compound is oxidized producing an aromatic carboxylic acid, and an aromatic amide compound. The aromatic amide compound is then hydrolyzed with a hydrolyzing agent to the aromatic carboxylic acid.

### BACKGROUND OF THE INVENTION

Oxidation of alkyl aromatic compounds, e.g., toluene and xylenes, are important commercial processes. A variety of oxidation products may be obtained including aromatic carboxylic acids such as terephthalic acid (1,4-benzenedicarboxylic acid) and isophthalic acid (1,3-benzenedicarboxylic acid) which are used, for example, in the polymer industry.

It is known that oxidation products, such as aromatic alcohols, aromatic aldehydes, aromatic ketones, and aromatic carboxylic acids, may solidify or crystallize at oxidation conditions and/or as the reaction mixture cools. Thus, mixtures of oxidation products may be produced which require further processing to increase the purity of the desired product. For example, in the production of terephthalic acid, the oxidation product is often referred to as crude terephthalic acid because it contains impurities including color bodies and intermediate oxidation products, especially 4-carboxybenzaldehyde (4-CBA). To obtain polymer grade or purified terephthalic acid, various purification steps are known in the art including: washing the crude terephthalic acid with water and/or a solvent, additional oxidation or crystallization steps, and reacting a solution of dissolved crude terephthalic acid with hydrogen at hydrogenation conditions usually including a catalyst comprising palladium and carbon. Often several purification steps are used.

U.S. Pat. No. 2,833,816 discloses processes for oxidizing aromatic compounds to the corresponding aromatic carboxylic acids. A process for the liquid phase oxidation of alkyl aromatic compounds uses molecular oxygen, a metal or metal ions, and bromine or bromide ions in the presence of an acid. The metals may include cobalt and/or manganese. Exemplary acids are lower aliphatic mono carboxylic acids containing 1 to 8 carbon atoms, especially acetic acid.

U.S. Pat. No. 6,355,835 discloses a process for the preparation of benzene dicarboxylic acids by liquid phase oxidation of xylene isomers using oxygen or air by oxidizing in the presence of acetic acid as a solvent, a cobalt salt as a catalyst, and an initiator. The oxidation step is followed by flashing the reaction mixture to remove volatile substances and cooling and filtering the material to get crude benzene di-carboxylic acid as a solid product and a filtrate. Recrystallizing the crude benzene di-carboxylic acid to obtain at least 99% purity and recycling of the filtrate are also disclosed.

U.S. Pat. No. 7,094,925 discloses a process for preparing an alkyl-aromatic compound. The process includes mixing an oxidizing agent or sulfur compound in the presence of an ionic liquid. Air, dioxygen, peroxide, superoxide, or any other form of active oxygen, nitrite, nitrate, and nitric acid or other oxides or oxyhalides of nitrogen (hydrate or anhydrous) can be used as the oxidizing agent. The process is typically carried out under Brønsted acidic conditions. The oxidation is preferably performed in an ionic liquid containing an acid promoter, such as methanesulfonic acid. The product is preferably a carboxylic acid or ketone or intermediate compound in the oxidation, such as an aldehyde, or alcohol.

U.S. Pat. No. 7,985,875 describes a process for preparing an aromatic polycarboxylic acid by liquid phase oxidation of a di- or tri-substituted benzene or naphthalene compound. The process involves contacting the aromatic compound with an oxidant in the presence of a carboxylic acid solvent, a metal catalyst, and a promoter in a reaction zone. The promoter is an ionic liquid comprising an organic cation and a bromide or iodide anion. The promoter is used in a concentration range of about 10 to about 50,000 ppm (based on solvent) with a preferred range of 10-1,000 ppm. No other promoters, such as bromine-containing compounds, need to be used in the process. The process produces crude terephthalic acid (CTA) having 1.4-2.2% 4-CBA. Purification of the CTA is required to obtain purified terephthalic acid (PTA).

US 2010/0174111 describes a process for purifying aryl carboxylic acids, such as terephthalic acid. The impure acid is dissolved or dispersed in an ionic liquid. A non-solvent (defined as a molecular solvent for which the ionic solvent has high solubility and for which the aryl carboxylic acid has little or no solubility) is added to the solution to precipitate the purified acid.

U.S. Pat. No. 7,692,036, 2007/0155985, 2007/0208193, and 2010/0200804 disclose a process and apparatus for carrying out the liquid-phase oxidation of an oxidizable compound. The liquid phase oxidation is carried out in a bubble column reactor that provides for a highly efficient reaction at relatively low temperatures. When the oxidized compound is para-xylene, the product from the oxidation reaction is CTA which must be purified. Purification is said to be easier than for conventional high temperature processes.

Recently, a method was disclosed to utilize a solvent comprising ionic liquid which significantly reduces the amount of 4-CBA in products. For example, US 2012/0004449, 2012/0004450, 2012/0004454, each of which is incorporated herein by reference, describe processes and mixtures for oxidizing alkyl aromatic compounds. The process involves forming a mixture comprising the alkyl-aromatic compound, a solvent, a bromine source, a catalyst, and optionally ammonium acetate, and contacting the mixture with an oxidizing agent at oxidizing conditions to produce an oxidation product comprising at least one of an aromatic aldehyde, and aromatic alcohol, an aromatic ketone, and an aromatic carboxylic acid. The solvent comprises a carboxylic acid having one to seven carbon atoms and an ionic liquid selected from an imidazolium ionic liquid, a pyridinium ionic liquid, a phosphonium ionic liquid, a tetra alkyl ammonium ionic liquid, or combinations thereof.

US 2012/0004456, which is incorporated herein by reference, describes a process for purifying crude terephthalic acid with a solvent comprising an ionic liquid at purifying conditions to produce a solid terephthalic acid product having a concentration of contaminant lower than the first concentration.

There is a need for improved processes for removing impurities in aromatic carboxylic acids.

### SUMMARY OF THE INVENTION

One aspect of the invention is a process for oxidizing an alkyl aromatic compound. In one embodiment, the process includes contacting the alkyl aromatic compound; an oxidation solvent comprising a carboxylic acid; ammonia or an ammonium compound; a bromine source; a catalyst; and an oxidizing agent in a reaction zone under oxidizing conditions to produce an oxidation product comprising an aromatic carboxylic acid, and an aromatic amide compound; and hydrolyzing the aromatic amide compound with a hydrolyzing agent to form an ammonium compound or ammonia and the aromatic carboxylic acid, wherein the hydrolyzing agent comprises an acid, or water, or both.

### BRIEF DESCRIPTION OF THE DRAWINGS

FIG. 1 is an illustration of the reaction sequence to form terephthalic acid from p-xylene.

FIGS. 2A-B are an illustration of one embodiment of a process to produce aromatic carboxylic acids incorporating hydrolysis of aromatic amides.

FIG. 3 is an illustration of another embodiment of a process to produce aromatic carboxylic acids incorporating hydrolysis of aromatic amides.

FIG. 4 is an illustration of another embodiment of an oxidation process to produce aromatic carboxylic acids incorporating hydrolysis of aromatic amides.

FIGS. 5A-B are graphs showing the amount of 4-CBA in the solid portion of a terephthalic acid product of para-xylene oxidation, as a function of the NH₃ equivalents.

FIG. 6 is a graph showing the amount of total aromatic amide in the solid portion of a terephthalic acid product of para-xylene oxidation, as a function of the NH₃ equivalents.

### DETAILED DESCRIPTION OF THE INVENTION

The invention generally involves the production of aromatic carboxylic acids from alkyl aromatic compounds. For ease of discussion, the application will refer to the production of terephthalic acid from p-xylene. However, it will be understood by those of skill in the art that other alkyl aromatic compounds and carboxylic acids could be used.

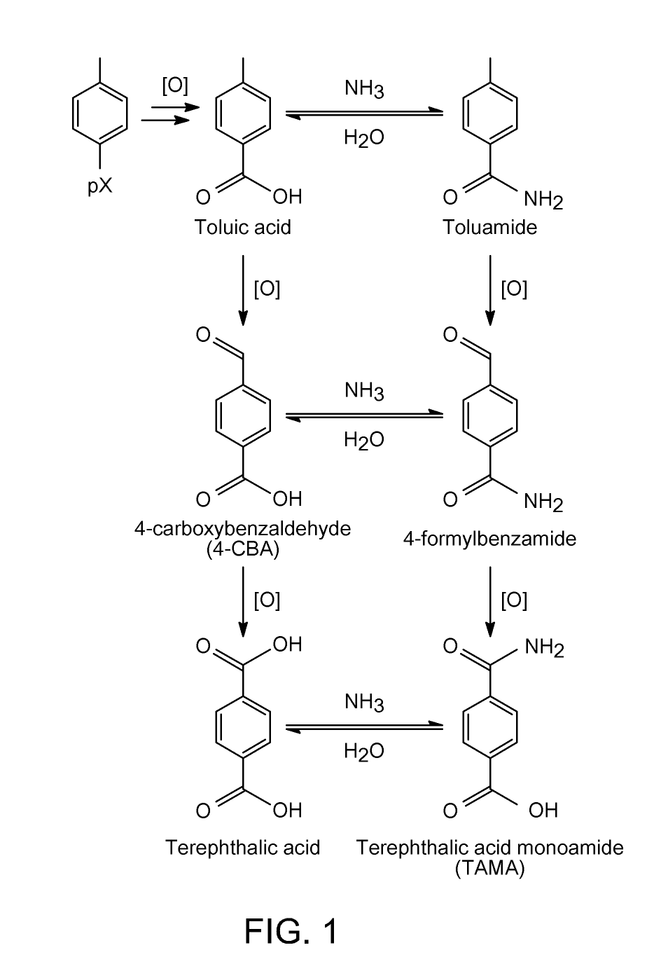

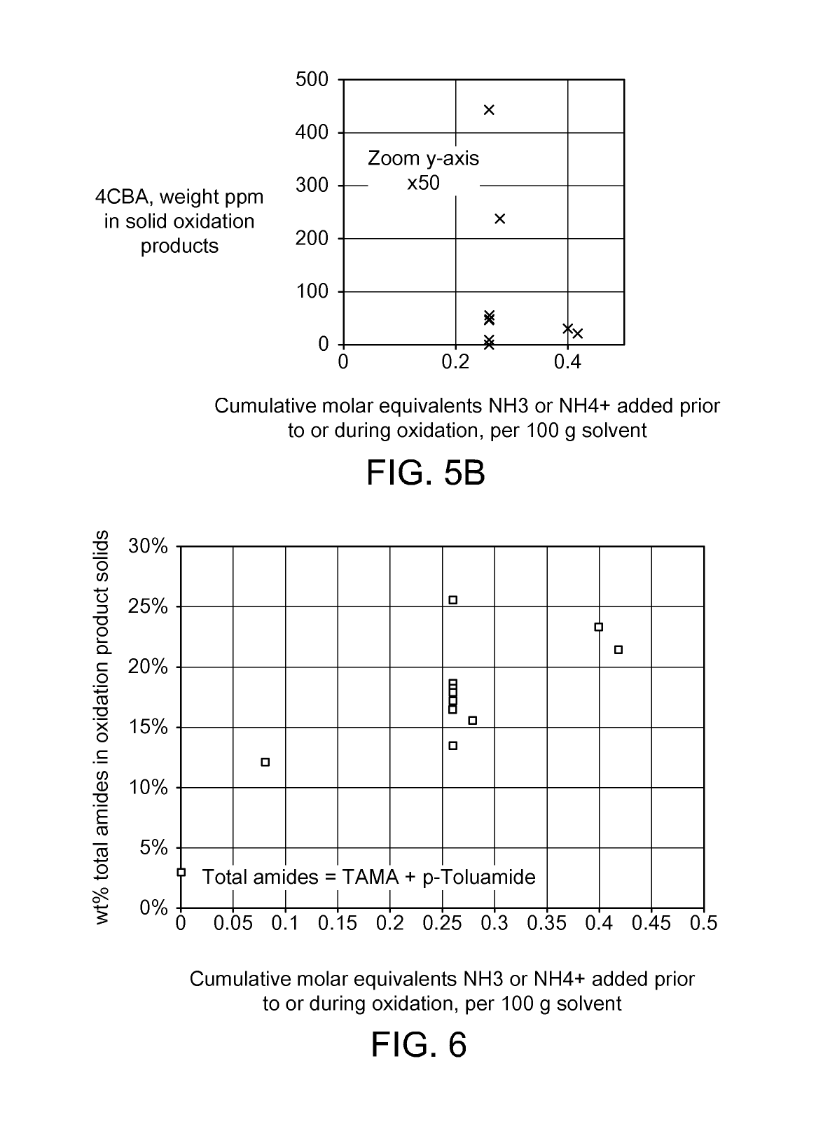

In the synthesis of terephthalic acid from p-xylene (see FIG. 1), a process utilizing a solvent comprising ionic liquid and acetic acid results in significantly reduced amounts of 4-CBA in the products. It has also been found that an additional reduction of the amount of 4-CBA in the solid products results when ammonium acetate, ammonia, or another ammonia source is added during reaction (see Tables 1 and 2 and FIGS. 5A-5B). The amount of 4-CBA in these solid products is sufficiently low that a hydrogenation stage, which is normally required to purify the crude product by converting 4-CBA to soluble toluic acid, can be avoided.

However, it was discovered that the introduction of ammonium compounds or ammonia into the reaction mixture results in the formation of amide derivatives of the aromatic carboxylic acid intermediates and products, notably p-toluamide and terephthalic acid monoamide (TAMA). These amides need to be removed or preferably converted to terephthalic acid for product to meet the specifications as a feed for polymer production.

Processes are known in which p-xylene is oxidized to terephthalic acid using acetic acid as a solvent (for instance, U.S. Pat. No. 5,081,290). There, the inclusion of ammonia or an ammonium compound in the reaction has been shown to provide some advantages such as reducing the amount of solvent burn under some conditions. Any amides formed in such processes would need to be removed.

The present invention includes an ammonium compound or ammonia in the p-xylene oxidation reaction to generate a composition of terephthalic acid containing low levels of 4-CBA, but unacceptably high levels of aromatic amides. The aromatic amides formed are then converted to aromatic carboxylic acids and ammonium compound or ammonia.

Solid oxidation products containing primarily terephthalic acid and terephthalic acid monoamide, and less than 0.5% toluic acid and toluamide, and less than 50 ppm 4-CBA with no detectable 4-formylbenzamide can be obtained using the oxidation portion of the present process. The terephthalic acid monoamide and toluamide are then converted to terephthalic acid and toluic acid respectively by hydrolysis.

The oxidation reaction products can be dissolved in the liquid phase at the reaction temperature, or they can be in the form of solids. In the presence of ammonia or ammonium compounds at high pressure and temperature, the oxidation reaction products undergo condensation with ammonia, resulting in the formation of an aromatic amide derivative of the aromatic carboxylic acid. The present process also includes conversion of the aromatic amide to the corresponding aromatic carboxylic acid by reversing the condensation reaction with hydrolysis. Hydrolysis can be accomplished by adding a hydrolyzing agent at elevated temperature (e.g., greater than about 150° C.). In the resulting hydrolyzed aromatic carboxylic acid product, the amount of aromatic amide is reduced to less than 7% of the amount of aromatic amide in the original oxidation product, and contains less than 2000 ppm nitrogen by mass.

Utilizing hydrolysis to convert amides to carboxylic acids has the added advantage of preventing yield loss. Rather than separating and removing the amide impurity, hydrolysis converts terephthalic acid monoamide to the desired product (terephthalic acid) and converts toluamide to p-toluic acid which is soluble under the hydrolysis conditions described in this invention and can be recycled to the reactor. Since no 4-formylbenzamide is generated in the oxidation portion of this process, hydrolysis does not result in additional 4-CBA.

In some embodiments, the hydrolysis can be performed in reactor vessels already required for reaction or crystallization in order to prevent significant added capital expense involved in an additional processing step which would otherwise require additional high pressure vessels. It can be performed at temperatures used in the existing processes. Examples of reactor design for the use of ionic liquids in p-xylene oxidation are described, for example, in US 2013/0041175, which is incorporated herein by reference.

The amide products can be hydrolyzed by the addition of a hydrolyzing agent. Suitable hydrolyzing agents include, but are not limited to, acids, water, a hydroxide capable of hydrolyzing the aromatic amide compound, or combinations thereof. Suitable acids include, but are not limited to, hydrobromic acid, hydrochloric acid, hydroiodic acid, toluenesulfonic acid, sulfuric acid, nitric acid, phosphoric acid, perchloric acid or carboxylic acids, such as acetic acid or formic acid, or combinations thereof. Addition of the hydrolyzing agent results in conversion of the amide products to form the corresponding carboxylic acid and ammonia or ammonium compound. The amount of acid in the hydrolyzing agent is typically about 10 to about 90 wt %, or about 70 to about 90 wt % if a carboxylic acid or other weak acid is used, or about 0.01 to about 10 wt %, or about 1 wt % to about 3 wt % if a strong acid is used. The amount of hydroxide is typically about 0.5 w % to about 10 wt %. The remainder of the hydrolyzing agent is typically water. The hydrolysis is the reverse of the condensation reaction responsible for forming the amides.

Hydrolysis can be carried out on reaction products which are partially or fully dissolved directly after the oxidation reaction and before crystallization, or during or after partial crystallization. It has also been demonstrated that hydrolysis is effective even on crystallized solid products, which are more difficult to hydrolyze due to the requirement to dissolve the solid.

In existing p-xylene oxidation processes, oxidation is followed by crystallization. In some cases, additional oxidation occurs in the crystallizers. Typically, several vessels are used for oxidation and crystallization. US Publication No. 2013/0041175 describes a flow scheme for use in ionic liquid based processes. In the current invention, hydrolysis is incorporated into a similar process flow scheme.

Hydrolysis can occur in several ways. In one embodiment, during the p-xylene oxidation reaction or immediately after conclusion of the oxidation reaction (where the oxidation reaction is stopped by discontinuing the source of oxidizing agent), the amide can be hydrolyzed by adding a hydrolyzing agent. This can take place in the oxidation zone. This is especially effective for semi-batch or plug flow reactor processes. In a batch or semi-batch process, the hydrolyzing agent can be added after the desired conversion to terephthalates is obtained. In a continuous flow process, such as a plug flow reactor, a stream of the hydrolyzing agent can be injected into the reactor at a location corresponding to the desired conversion. In a process utilizing a continuous stirred tank reactor, a similar concept could be employed, except that the injection of the hydrolyzing agent would occur in a separate vessel from the oxidation vessel. The effluent of the oxidation reactor would flow into such a hydrolysis vessel and be contacted with hydrolysis agent.

In another embodiment, a solvent which is effective for both hydrolysis and crystallization can be added during crystallization in a separate vessel or vessels from the oxidation reaction vessel. The hydrolysis/crystallization solvent generally contains water and/or an acid capable of hydrolyzing the amides. In addition, the product, e.g., terephthalic acid, should be less soluble in the hydrolysis/crystallization solvent than in the reaction solvent. Water and/or acetic acid are suitable examples of hydrolysis/crystallization solvents. If the hydrolysis/crystallization solvent is added at a temperature up to about 100° C. higher than, or up to about 20° C. lower than the reaction temperature, hydrolysis will take place during contacting of the product stream with the hydrolysis/crystallization solvent. In this way, hydrolysis takes place on an amide-containing product that is at least partially still dissolved in the liquid phase. Crystallization then proceeds by lowering the temperature and/or changing the solvent composition to decrease terephthalic acid solubility. For instance, terephthalic acid is less soluble in acetic acid than in water. This can be done in the same or a successive vessel. The spent crystallization solvent, which contains dissolved ammonia, can optionally be recycled and used as part of the make-up oxidation reactor solvent in a continuous process.

In another embodiment, an additional process step is added in which the solid terephthalic acid composition containing amides is hydrolyzed after crystallization. This can be accomplished for instance by heating at high temperature and pressure (sufficient to prevent boiling) in a carboxylic acid, such as acetic acid or a carboxylic acid-water mixture. The temperature used range from about 180° C. to about 300° C., or from about 190° C. to about 230° C., or from about 200° C. to 215° C. Temperatures at the higher end of these ranges are used in order to dissolve the solid more extensively and to allow for operation with a higher solid/solvent ratio. Lower temperatures are used decrease utility costs. To obtain low levels of nitrogen containing compounds, this mixture is then filtered hot (e.g., at about 90° C. to about 200° C.) to remove the solvent and the dissolved ammonia which results from the hydrolysis. If higher temperature is utilized for the hydrolysis, the temperature can be decreased to below about 200° C. to avoid loss of terephthalic acid in the filtrates. Optionally, the product is then washed with a hydrolyzing agent at a lower temperature (e.g., about 90° C. to about 125° C.) to remove ammonium salts.

Hydrolysis is effective at converting all or most of the aromatic amide compounds and removing all or most of the nitrogen containing products such as ammonia and/or nitrogen containing salts that would be expected to form from ammonia or an ammonium compound. For example, the amount of aromatic amide compound after hydrolysis can be less than about 7% of the amount of aromatic amide compound by mass in the oxidation product, or less than about 5%, or less than about 4%, or less than about 2%, or less than about 1%, or less than about 0.5%. The nitrogen content in the aromatic carboxylic acid after hydrolysis can be less than about 2000 ppm (by mass), or less than about 1000 ppm, or less than about 200 ppm. The nitrogen content in the aromatic carboxylic acid after hydrolysis can be less than about 5% of the amount of nitrogen (by mass) in the oxidation product, or less than about 3%, or less than about 2.5%.

In one embodiment, the oxidation conditions include a p-xylene to solvent ratio of 1:5 by weight, a temperature of about 215° C. and a pressure of 2.758 MPa (400 psig) for about 3 hours under flowing air bubbled through a semi-continuous batch reactor at 2-2.5 L/min, where the solvent comprises about 49 to about 62 wt % acetic acid, about 30 to about 38 wt % ionic liquid, and either 20 wt % ammonium acetate or 2.5 vol % ammonia included in the air stream. Under these conditions, solid products containing primarily terephthalic acid and terephthalic acid monoamide, with less than 0.5% toluic acid and toluamide, less than 50 ppm 4-CBA, and no detectable 4-formylbenzamide were obtained.

The reactions can take place in batch reactors, semi-batch reactors, or continuous reactors, such as continuously stirred tank reactors or plug flow reactors.

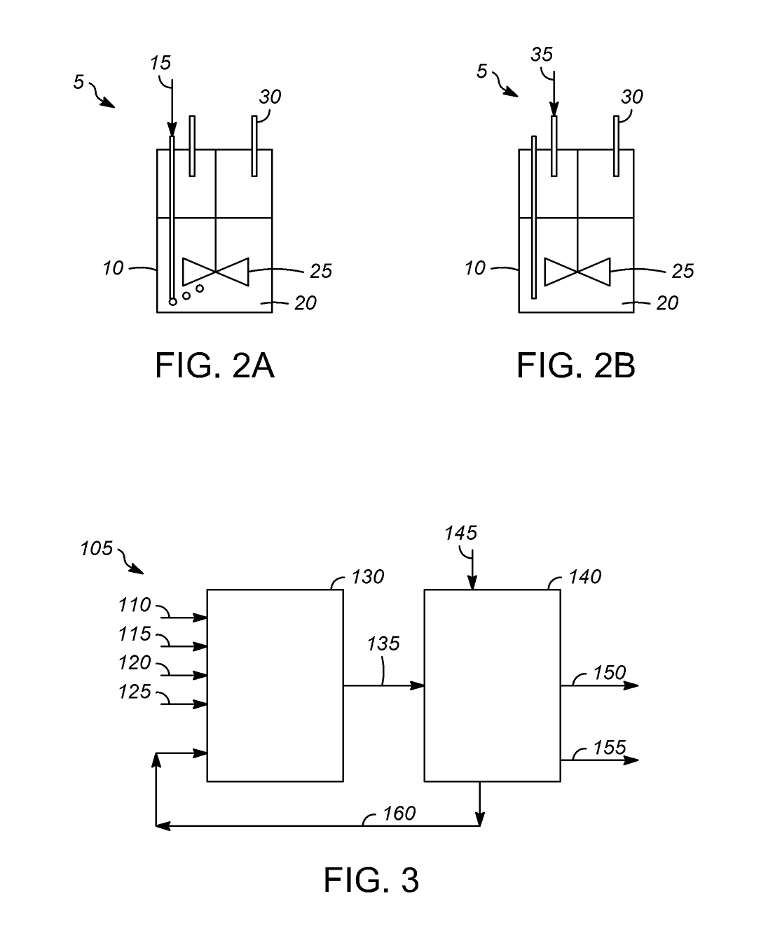

FIGS. 2A-B illustrate one embodiment of a semi-batch process 5 utilizing a single reactor 10. As shown in FIG. 2A, the oxidation reaction takes place in oxidation reactor 10. The p-xylene, solvent, ammonia or an ammonium compound, a bromine source, and a catalyst are added to reactor 10. The oxidizing agent 15, such as air, is introduced into the reactor 10. The reaction mixture 20 can be stirred using a mixer 25. The gaseous reaction products and unreacted gases are vented out a vent 30.

In some embodiments, the reaction solvent further comprises at least one ionic liquid. Two or more ionic liquids can be used, if desired.

When ionic liquids are used in the oxidation, the oxidation reactor 10 is typically operated at a temperature of about 240° C. or less, or in a range of about 170° C. to about 240° C., or about 190° C. to about 220° C. when the oxidation takes place in the presence of a solvent including an ionic liquid. When the oxidation takes place in the absence of an ionic liquid, it is typically operated at a temperature of about 210° C. or less, or in a range of about 160° C. to 200° C., or in a range of about 170° C. to 190° C. The pressure is generally in a range of about 0.69 MPa(g) (100 psig) to about 4.1 MPa(g) (600 psig), or about 1.4 MPa(g) (200 psig) to about 2.8 MPa(g) (405 psig). The residence time is typically about 60 to about 180 minutes when the solvent includes ionic liquid, and about 5 to about 60 minutes when it does not.

The reaction proceeds until a desired conversion to terephthalic acid is obtained. The addition of the oxidation agent 15 is then stopped.

The hydrolyzing agent 35 is added to the reactor 10, as shown in FIG. 2B. The hydrolyzing agent 35 should be effective at hydrolyzing the amide compounds formed during oxidation to their corresponding acids and crystallizing the terephthalic acid. The hydrolyzing agent can include water, acid, or a hydroxide capable of hydrolyzing the amides. Suitable hydrolyzing agents include, but are not limited to, water, carboxylic acids such as acetic acid, strong acids such as hydrobromic acid in water, and hydroxides such as sodium hydroxide in water. The product is then crystallized by cooling the reactor. The crystallized terephthalic acid can then be recovered using known separation processes, including, but not limited to, filtration, centrifugation, decantation, and the like.

FIG. 3 illustrates one embodiment of a continuous process 105. P-xylene 110, oxidant 115, oxidation solvent 120, and ammonia or an ammonium compound 125 are introduced into oxidation reaction zone 130 where the p-xylene 110 is oxidized to terephthalic acid.

The oxidation conditions are similar to those described above.

Additional components can be included with the oxidation solvent, such as the bromine source, and the catalyst, if desired.

The effluent 135 from the oxidation reaction zone 130 is sent to a hydrolysis/crystallization zone 140. Hydrolysis is initiated by adding a hydrolyzing agent 145 to the hydrolysis/crystallization zone 140. The hydrolyzing agent 145 is a solvent which is effective for both hydrolysis and crystallization. It typically contains water and an acid capable of hydrolyzing the amides, and the terephthalic acid product has a lower solubility in it compared to the oxidation solvent. Examples of hydrolyzing agents include water and carboxylic acids, such as acetic acid.

The temperature of the hydrolysis/crystallization zone 140 is generally at least about 150° C., or at least about 200° C. The pressure is sufficient to maintain the solvent in the liquid phase at the chosen temperature. The pressure is generally at least about 0.27 MPa (g) (40 psig) or at least about 0.76 MPa (g) (110 psig).

Crystallization can be enhanced by lowering the temperature in the hydrolysis/crystallization zone 140, if desired.

Solid terephthalic acid 150 can be separated from the reaction mixture and recovered.

In some embodiments, a portion 155 of the reaction mixture can be sent to one or more crystallization zones (not shown), if desired.

A portion 160 of the reaction mixture can be recycled to the oxidation reaction zone 130. Other components can be added fresh, or from other sources to the recycle portion 160 to obtain a proper composition for recycle to the oxidation reaction zone 130. This mixture also contains the hydrolyzing agent and the ammonia or ammonium compound that resulted from hydrolysis. If recycled, these components serve as a portion of the solvent and ammonia or ammonium compound in the oxidation zone 130.

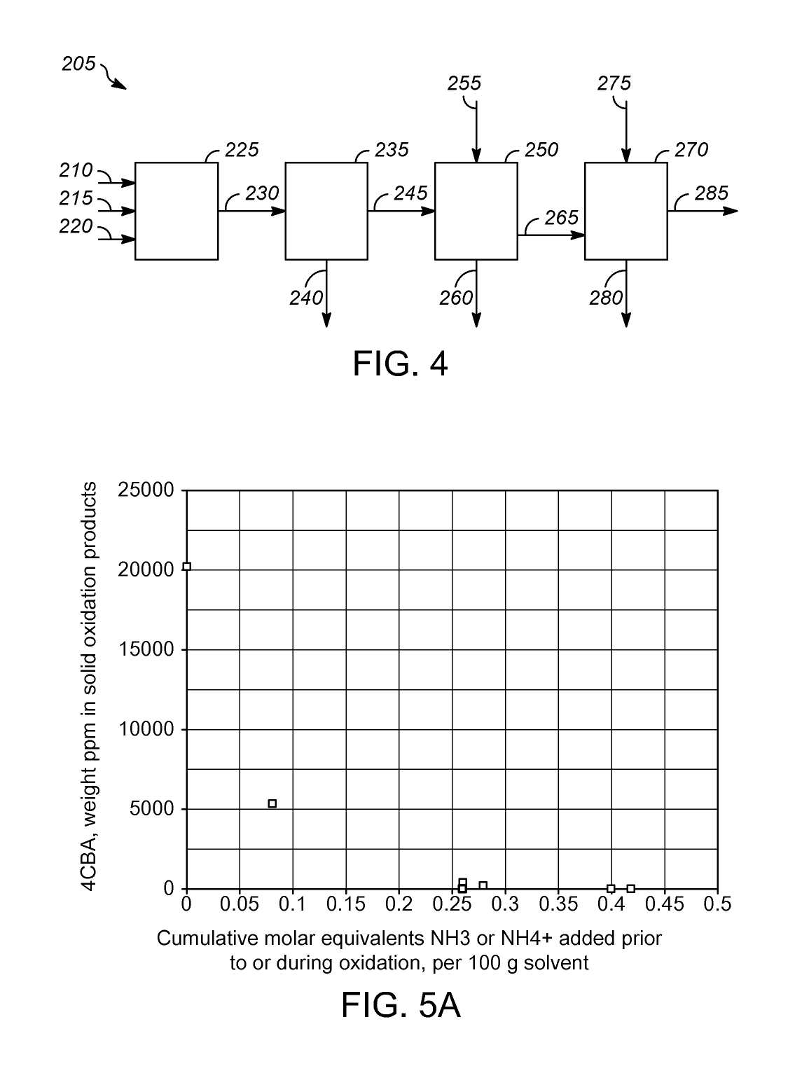

FIG. 4 illustrates another embodiment in which a solid product is hydrolyzed. In the process 205, p-xylene 210, oxidant 215, and solvent 220, are introduced into zone 225 where the oxidation of p-xylene and the crystallization of the terephthalic acid reaction product take place. Depending on the process used, ammonia or an ammonium compound can also be included. One or more reaction and/or crystallization zones can comprise zone 225.

The effluent 230 from zone 225 is sent to separation zone 235 where the liquid 240 is separated from the solid product 245. The liquid 240 can be sent for solvent treatment or recycling.

The solid product 245 is sent to hydrolysis zone 250. A hydrolyzing agent 255 is added to the hydrolysis zone 250 where it hydrolyzes the amide compounds in the solid product 245. The used hydrolyzing agent 260 can be sent for solvent treatment or recycle.

Optionally, the solid product 265 from the hydrolysis zone 250 is sent to wash zone 270 where a washing solvent 275 is introduced to wash the solid product 265. The used solvent 280 can be sent for treatment or recycled. The washed solid product 285 can be recovered.

The process can be used for the oxidation of alkyl aromatic compounds to aromatic carboxylic acids. Suitable alkyl aromatic compounds or feeds to be oxidized include aromatic compounds comprising at least one benzene ring having at least one alkyl group. Methyl, ethyl, and isopropyl alkyl groups are preferred alkyl groups, although other alkyl groups can be used if desired. In an embodiment, the alkyl aromatic compound is selected from toluene, para-xylene, ortho-xylene, and meta-xylene. The feed may comprise more than one alkyl aromatic compound. As the oxidation reaction generally proceeds through successive degrees of oxidization, suitable feed compounds also include partially oxidized intermediates relative to the desired oxidized product such as para-, meta-, or ortho-toluic acid.

In some embodiments, the reaction solvent includes ionic liquids. Generally, ionic liquids are non-aqueous, organic salts composed of ions where the positive ion is charge balanced with a negative ion. These materials have low melting points, often below 100° C., undetectable vapor pressure, and good chemical and thermal stability. The cationic charge of the salt is localized over hetero atoms, and the anions may be any inorganic, organic, or organometallic species.

In an embodiment, ionic liquids suitable for use include, but are not limited to, one or more of imidazolium ionic liquids, pyridinium ionic liquids, tetra alkyl ammonium ionic liquids, and phosphonium ionic liquids. More than one ionic liquid may be used. Imidazolium, pyridinium, and ammonium ionic liquids have a cation comprising at least one nitrogen atom. Phosphonium ionic liquids have a cation comprising at least one phosphorus atom. In an embodiment, the ionic liquid comprises a cation selected from alkyl imidazolium, di-alkyl imidazolium, and combinations thereof. In another embodiment, the ionic liquid comprises an anion selected from halides, acetate, carboxylates, and combinations thereof. The ionic liquid may comprise at least one of 1-butyl-3-methylimidazolium acetate (BMImOAc), 1-butyl-3-methylimidazolium bromide (BMImBr), 1-hexyl-3-methylimidazolium acetate (C6MImOAc), and 1-hexyl-3-methylimidazolium bromide (C6MImBr).

The ionic liquid can be provided, or it can be generated in situ from appropriate precursors, or both.

In some embodiments, the reaction solvent has a ratio of the ionic liquid solvent to the carboxylic acid solvent within a range of about 0.1:1 to about 10:1 by weight, or about 0.1:1 to about 7:1, or about 0.1:1 to about 5:1, or about 0.1:1 to about 3:1. The amount of ionic liquid includes ionic liquid precursors, if present. The optional ionic solid or material capable of forming an ionic salt in solution discussed below, if present, is included in the amount of ionic liquid.

The oxidation reaction mixture typically includes a catalyst. The catalyst generally comprises at least one of cobalt, manganese, titanium, chromium, copper, nickel, vanadium, iron, molybdenum, tin, cerium and zirconium. In an embodiment, the catalyst comprises cobalt and manganese. The metal may be in the form of an inorganic or organic salt. For example, the metal catalyst may be in the form of a carboxylic acid salt, such as, metal acetate and hydrates thereof. Exemplary catalysts include cobalt (II) acetate tetrahydrate and manganese (II) acetate, individually or in combination. In an embodiment, the amount of manganese (II) acetate is less than the amount of cobalt (II) acetate tetrahydrate by weight.

The amount of catalyst used may vary widely. For example, the amount of cobalt may range from about 0.001 wt % to about 2 wt % relative to the weight of the solvent. In an embodiment, the amount of cobalt ranges from about 0.05 wt % to about 2 wt % relative to the weight of the solvent. The amount of manganese may range from about 0.001 wt % to about 2 wt % relative to the weight of the solvent. In an embodiment, the amount of manganese ranges from about 0.05 wt % to about 2 wt % relative to the weight of the solvent. In another embodiment, the ratio of cobalt to manganese ranges from about 3:1 to about 1:2 by weight on an elemental metal basis.

The oxidation reaction mixture typically includes a bromine source. Bromine sources are generally recognized in the art as being catalyst promoters and include bromine, ionic bromine, e.g. HBr, NaBr, KBr, NH₄Br; and/or organic bromides which are known to provide bromine at the oxidation conditions, such as, benzylbromide, mono and di-bromoacetic acid, bromoacetyl bromide, tetrabromoethane, ethylene di-bromide. In an embodiment, the bromine source comprises or consists essentially of or consists of hydrogen bromide. The amount of hydrogen bromide may range from about 0.01 wt % to about 5 wt %, relative to the weight of the solvent, or about 0.01 wt % to about 4 wt %, or about 0.01 wt % to about 3 wt %, or about 0.01 wt % to about 2 wt %, or about 0.01 wt % to about 1.5 wt %, or about 0.01 wt % to about 1 wt %. In another embodiment, the amount of hydrogen bromide ranges from about 0.25 wt % to about 0.75 wt %, relative to the weight of the solvent. The solvent includes the carboxylic acid, the ionic liquid, the ionic liquid precursors, the optional ionic solid or material capable of forming an ionic salt in solution, and the optional water.

Suitable oxidizing agents for the process provide a source of oxygen atoms to oxidize the p-xylene and/or p-toluic acid, and/or another intermediate oxidization product at the oxidation conditions employed. Examples of oxidizing agents include peroxides, superoxides, and nitrogen compounds containing oxygen such as nitric acids. In an embodiment, the oxidizing agent is a gas comprising oxygen, e.g. air, and molecular oxygen. The gas may be a mixture of gases. The amount of oxygen used in the process is preferably in excess of the stoichiometric amount required for the desired oxidation process. In an embodiment, the amount of oxygen contacted with the mixture ranges from about 1.2 times the stoichiometric amount to about 100 times the stoichiometric amount. Optionally, the amount of oxygen contacted with the mixture may range from about 2 times the stoichiometric amount to about 30 times the stoichiometric amount.

The amount of ammonia or ammonium compound added to the reaction zone is chosen such that concentration of ammonia or ammonium compound is about 0.5 to about 5 mol/L of solvent, or preferably about 2 to about 3 mol/L. Alternatively, the amount of ammonia or ammonium compound added to the reaction zone is chosen such that the pH of the reaction medium (measured at room temperature) is between about 3 and about 7, or preferably between about 3.5 and about 5. The ammonia or ammonium can be added in the form of anhydrous ammonia in the gas stream, added together with the oxidizing agent if a gaseous oxidizing agent is used. The concentration of ammonia in the gas stream is between about 0.25 vol % and about 3 vol %. Alternatively, an ammonium source is added to the reaction solvents. Examples of suitable ammonium compounds are ammonium acetate, ammonium carbonate, and ammonium bromide.

The process may be practiced in laboratory scale experiments through full scale commercial operations. The process may be operated in batch, continuous, or semi-continuous mode. The contacting step can take place in various ways. The order of addition of the components (e.g., alkyl-aromatic compound, solvent, bromine source, catalyst, and oxidizing agent) is not critical. For example, the components can be added individually, or two or more components may be combined or mixed before being combined or mixed with other components. Individual process steps may be operated continuously and/or intermittently as needed for a given embodiment, e.g., based on the quantities and properties of the streams to be processed in such steps.

As used herein, the term “zone” can refer to one or more equipment items and/or one or more sub-zones. Equipment items may include, for example, one or more vessels, heaters, separators, exchangers, conduits, pumps, compressors, and controllers. Additionally, an equipment item can further include one or more zones or sub-zones. The solids separation, and/or contaminant extraction process or step may be conducted in a similar manner and with similar equipment as is used to conduct other solid-liquid wash and separation operations.

Suitable equipment for solids separation includes, for example, filters, mixers, stirred tanks, settlers, centrifuges, cyclones, decanters, and augers. Suitable equipment for contaminant removal from liquid streams includes columns with trays, packing, rotating discs or plates, distillation columns, and vacuum distillation vessels.

### Comparative Example

### No Ammonium Acetate

20 g para-xylene was oxidized in a mixture of 50 g acetic acid, 20 g BMImBr, 10 g BMImOAc, and 0.4 g water with catalyst composed of 0.8 g Cobalt(II) acetate tetrahydrate, 0.6 g manganese(II) acetate and 0.4 g Hbr. The reaction took place at 215° C. for three hours in semi-batch mode with continuous flow of air at 2500 sccm, and the temperature was maintained for the duration. After replacing air with nitrogen and cooling to room temperature to achieve crystallization, the product was filtered to remove solid reaction products. Note that room temperature filtration does not separate toluic acid from terephthalic acid, although filtration above 180° C. would. The solids were washed 3 times with room temperature water and then stirred in water at 80° C. for 30 min and filtered hot. The solid terephthalic acid reaction products produced in this way had 4-CBA content of 20278 ppm, and toluic acid content of 2295 ppm. On two occasions p-toluamide content was measured and was 1106 and 5797 ppm respectively. The results are shown in Table 1. The 4-CBA content of the solid products for this example and examples 1 and 2, as well as the total amide content of the solid products (toluamide+terephthalic acid monoamide) are also shown in FIGS. 5 and 6 respectively. There, the data are plotted as a function of the cumulative amount of ammonium acetate or ammonia in moles that were added to the reactor.

### Example 1

### Oxidation in the Presence of Ammonium Acetate to Generate Amide Containing Products

In one set of examples, 20 g para-xylene was oxidized in a mixture of 50 g acetic acid, 20 g BMImBr, 10 g BMImOAc, 20 g ammonium acetate, and 0.4 g water with catalyst composed of 0.8 g Cobalt(II) acetate tetrahydrate, 0.6 g manganese(II) acetate and 0.4 g HBr. The reaction took place at 215° C. for three hours in semi-batch mode with continuous flow of air at 2500 sccm, and the temperature was maintained for the duration. After replacing air with nitrogen and cooling to room temperature to achieve crystallization, the product was filtered to remove solid reaction products. Note that room temperature filtration does not separate toluic acid from terephthalic acid, although filtration above 180° C. would. The solids were washed 3 times with room temperature water and then stirred in water at 80° C. for 30 min and filtered hot. The solid terephthalic acid reaction products produced in this way (seven repetitions, examples 1A-1E) had an average 4-CBA content of 19.7 ppm, an average toluic acid content of 2475 ppm, and an average terephthalic acid monoamide content of 18%. On two occasions p-toluamide content was measured and was 1106 and 5797 ppm respectively. The detailed data is shown in Table 1. The 4-CBA content of the solid products for this example, the comparative example and example 2, as well as the total amide content of the solid products (toluamide+terephthalic acid monoamide) are also shown in FIGS. 5 and 6 respectively. There, the data are plotted as a function of the cumulative amount of ammonium acetate or ammonia in moles that were added to the reactor.

### Example 2

### Oxidation in the Presence of Ammonia Gas Feed to Generate Amide Containing Products

In another set of examples, ammonia was introduced in the gas phase instead of including ammonium acetate. In all of these examples, the catalyst was composed of 0.8 g Cobalt(II) acetate tetrahydrate, 0.6 g manganese(II) acetate and 0.4 g BBL The reaction took place at 215° C. for three hours in semi-batch mode with continuous flow of air/ammonia mixture at 2500 sccm, and the temperature was maintained for the duration. After replacing air with nitrogen and cooling to room temperature to achieve crystallization, the product was filtered to remove solid reaction products. The solids were washed 3 times with room temperature water and then stirred in water at 80° C. for 30 min and filtered hot. The detailed data is shown in Table 2. The 4-CBA content of the solid products for this example, the comparative example and example 1, as well as the total amide content of the solid products (toluamide+terephthalic acid monoamide) are also shown in FIGS. 5 and 6 respectively. There, the data are plotted as a function of the cumulative amount of ammonium acetate or ammonia in moles that were added to the reactor.

In example 2A, 20 g para-xylene was oxidized in a mixture of 62.5 g acetic acid, 25.1 g BMImBr, 12.6 g BMImOAc, and 0.4 g water. The air/ammonia mixture contained 0.5 volume % ammonia. The solid terephthalic acid reaction products produced in this had 4-CBA content of 5401 ppm, toluic acid content of 598 ppm, toluamide content of 2221 ppm and terephthalic acid monoamide content of 11.9%.

In example 2B, 20 g para-xylene was oxidized in a mixture of 62.5 g acetic acid, 25 g BMImBr, 12.6 g BMImOAc, and 0.4 g water. The air/ammonia mixture contained 2.5 volume % ammonia. The solid terephthalic acid reaction products produced in this had 4-CBA content of 22 ppm, toluic acid content of 536 ppm, toluamide content of 5666 ppm and terephthalic acid monoamide content of 20.9%.

In example 2C, 20 g para-xylene was oxidized in a mixture of 66.3 g acetic acid, 20 g BMImBr, 10.1 g BMImOAc, and 0.4 g water. An air/ammonia mixture containing 2.5 volume % ammonia was flowed for 2 hours, and then air with no ammonia was flowed for 1 hour. The solid terephthalic acid reaction products produced in this had 4-CBA content of 238 ppm, toluic acid content of 0 ppm, toluamide content of 6578 ppm and terephthalic acid monoamide content of 14.9%.

In example 2D, 20 g para-xylene was oxidized in a mixture of 50 g acetic acid, 20 g BMImBr, 10.5 g BMImOAc, 20 g ammonium acetate and 0.4 g water. Air was flowed for 2 hours, followed by an air/ammonia mixture containing 2.5 volume % ammonia flowed for 1 hour. The solid terephthalic acid reaction products produced in this had 4-CBA content of 31 ppm, toluic acid content of 871 ppm, toluamide content of 376 ppm and terephthalic acid monoamide content of 23.3%.

### Example 3

### Hydrolysis of Amide Containing Products with Mixture of Acetic Acid and Water

Aromatic carboxylic acids and amides were generated by oxidizing p-xylene in the presence of ammonium acetate acetic acid and ionic liquid in a procedure identical to Example 1, to produce amide containing terephthalic acid products.

Hydrolysis of the amide-containing product was accomplished by heating the solid composition to 200° C. and 2.758 MPa(g) (400 psig) in a mixture containing 80% acetic acid and 20% water. The high temperature step was followed by a lower temperature wash (95° C.) of the crystallized product, also using 80% acetic acid and 20% water. This hydrolysis treatment reduced the amount of nitrogen in the solid product from 4.25% after the oxidation reaction to 0.34% after the high temperature treatment, and further to undetectable nitrogen content after the 95° C. acetic acid wash, with no mass loss. HPLC analysis of these samples showed that the TAMA content was reduced from 30.5% in the original oxidation product to 2% in the hydrolysis product, and the toluamide content was reduced from 1.7% to undetectable.

Similar treatments were also performed using only water or only acetic acid as the solvents (although these started with oxidation products which contained different amounts of amides as shown in Table 3), and using a 20% water, 80% acetic acid mixture at 150° C. instead of 200° C. The amount of nitrogen in the solid products was reduced, but to a lesser extent (see Table 3).

These examples were conducted in a closed system and cooled to room temperature, providing a worst-case scenario since nitrogen-containing products may crystallize with the terephthalic acid.

### Example 4

### Hydrolysis of Amide Containing Products with Mixture of Hydrobromic Acid (HBr) and Water

2 grams of solid para-xylene oxidation product containing primarily terephthalic acid with amide compounds was combined with an aqueous solution of HBr. The solution contained 0.25, 0.5, or 0.75 g of HBr in 50 grams of water. The mixture was heated to 200° C. in a sealed autoclave and was held at that temperature for 15 minutes. The product was cooled, filtered, and washed with water. When 0.25 g HBr was used, the isolated solid contained 1.0% nitrogen. When 0.5 g HBr was used, the isolated solid contained 0.35% nitrogen. When 0.75 g HBr was used the isolated solid contained 0.07% nitrogen. When 1.0 g HBr was used, the isolated solid contained no detectable nitrogen.

TABLE 1

| | | NH3 or | | Solid components | | | | | | | | |
|---|---|---|---|---|---|---|---|---|---|---|---|---|
| | | NH4OAc | | | p- | | | | | | | |
| | | moles/ | Solid | p- | toluic | 4- | | | benzoic | | | Total |
| Solvent and | | 100 g | weight | toluamide | acid | CBA | % | % | acid | wt % | Total | toluates |
| gas feed | Sample | solvent | (g) | ppm | ppm | ppm | TA-ma | TA | ppm | Amides | terephthalates | ppm |
| No | Comparative | 0 | 26.86 | 0 | 2295 | 20278 | 3.0%* | 86.3% | 502 | 3% | 89.3% | 2295 |
| ammonium | example | | | | | | | | | | | |
| acetate or | | | | | | | | | | | | |
| ammonia | | | | | | | | | | | | |
| Solvent with | 1A | 0.26 | 25.13 | not | 310 | 0 | 18.3% | 77.3% | 0 | 18.3% | 95.6% | 310 |
| 20 wt % | | | | analyzed | | | | | | | | |
| ammonium | 1B | 0.26 | 19.8 | not | 196 | 0 | 13.5% | 79.4% | 0 | 13.5% | 92.9% | 196 |
| acetate, gas | | | | analyzed | | | | | | | | |
| feed air only | 1C | 0.26 | 19.97 | 5797 | 11051 | 49.1 | 25.0% | 76.1% | 3733 | 25.6% | 101.1% | 16848 |
| | 1D | 0.26 | 20.78 | 1106 | 4852 | 23.5 | 18.4% | 75.6% | 2116 | 18.5% | 93.9% | 5959 |
| | 1E | 0.26 | 17.9 | not | 160 | 0 | 16.5% | 77.7% | 3830 | 16.5% | 94.2% | 160 |
| | | | | analyzed | | | | | | | | |
| | 1F | 0.26 | 18.22 | not | 658 | 55.8 | 17.9% | 76.1% | 0 | 17.9% | 94.0% | 658 |
| | | | | analyzed | | | | | | | | |
| | 1G | 0.26 | 19.8 | not | 94 | 9.3 | 17.2% | 76.2% | 0 | 17.2% | 93.5% | 94 |
| | | | | analyzed | | | | | | | | |
| | | | mean | | 2475 | 19.7 | 18% | 77% | 1383 | 18% | 95% | 3461 |
| | | □ | □ | | 4152 | 24 | 3.5% | 1.3% | 1812 | 4% | 2.8% | 6273 |

TABLE 2

| | | | | | | | | | | | | |
|---|---|---|---|---|---|---|---|---|---|---|---|---|
| No | 2A | 0.08 | 25.98 | 2221 | 598 | 5401 | 11.9% | 85.6% | 2700 | 12% | 97.5% | 2819 |
| ammonium in | | | | | | | | | | | | |
| solvent, | | | | | | | | | | | | |
| 0.5% v NH3 | | | | | | | | | | | | |
| in gas feed | | | | | | | | | | | | |
| No | 2B | 0.42 | 26.2 | 5666 | 536 | 22 | 20.9% | 72.5% | 2465 | 21% | 93.4% | 6202 |
| ammonium in | | | | | | | | | | | | |
| solvent, | | | | | | | | | | | | |
| 2.5% v NH3 | | | | | | | | | | | | |
| in gas feed | | | | | | | | | | | | |
| No | 2C | 0.28 | 25.86 | 6578 | 0 | 238 | 14.9% | 77.0% | 3744 | 16% | 91.9% | 6578 |
| ammonium in | | | | | | | | | | | | |
| solvent, | | | | | | | | | | | | |
| 2.5% v NH3 | | | | | | | | | | | | |
| in gas feed | | | | | | | | | | | | |
| 2 hrs, then 1 | | | | | | | | | | | | |
| hour air only | | | | | | | | | | | | |
| Solvent with | 2D | 0.4 | 26.6 | 376 | 871 | 31 | 23.3% | 73.6% | 0 | 23% | 96.9% | 1247 |
| 20 wt % | | | | | | | | | | | | |
| ammonium | | | | | | | | | | | | |
| acetate. Gas | | | | | | | | | | | | |
| feed 2 hour | | | | | | | | | | | | |
| air only, then | | | | | | | | | | | | |
| 2.5% v NH3 | | | | | | | | | | | | |
| in air | | | | | | | | | | | | |

*The HPLC method used to measure composition of solid products used ammonium hydroxide as solvent which caused a baseline signal corresponding to 0-3% to be present in any sample containing terephthalic acid due to room temperature reaction in the sample.

TABLE 3

| | Before Wash | | | 1^{st} Cycle | | | 2^{nd} Cycle | | |
|---|---|---|---|---|---|---|---|---|---|
| | Ppm | % | % | Ppm | % | % | Ppm | % | % |
| | 4CBA | TAMA | N | 4CBA | TAMA | N | 4CBA | TAMA | N |
| 20% | 38 | 30.5% | 4.25 | 54 | Not | 0.34 | 0 | 2.0% | <0.10 |
| water | | | | | measured | | | | |
| 80% | | | | | | | | | |
| Acetic | | | | | | | | | |
| Acid | | | | | | | | | |
| 200 C. | | | | | | | | | |
| Water | 86 | Not | 4.57 | 61 | Not | 1.25 | 54.53 | Not | 1.16 |
| Wash | | measured | | | measured | | | measured | |
| 200 C. | | | | | | | | | |
| Acetic | 86 | Not | 4.57 | 22 | Not | 0.64 | 30.61 | Not | 0.69 |
| Acid | | measured | | | measured | | | measured | |
| Wash | | | | | | | | | |
| 200 C. | | | | | | | | | |
| 20% | 7519 | 15.9% | 0.99 | 5800 | 13.4% | 0.76 | — | — | — |
| water | | | | | | | | | |
| 80% | | | | | | | | | |
| Acetic | | | | | | | | | |
| Acid | | | | | | | | | |
| 150 C. | | | | | | | | | |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A process for oxidizing an alkyl aromatic compound comprising:
contacting the alkyl aromatic compound; an oxidation solvent comprising a carboxylic acid; ammonia or an ammonium compound; a bromine source; a catalyst; and an oxidizing agent in a reaction zone under oxidizing conditions to produce an oxidation product comprising an aromatic carboxylic acid, and an aromatic amide compound; and
hydrolyzing the aromatic amide compound with a hydrolyzing agent to form an ammonium compound or ammonia and the aromatic carboxylic acid, wherein the hydrolyzing agent comprises an acid, water, a hydroxide, or combinations thereof.

2. The process of claim 1 wherein the acid in the hydrolyzing agent comprises a carboxylic acid.

3. The process of claim 1 wherein the acid in the hydrolyzing agent comprises hydrobromic acid, hydrochloric acid, hydroiodic acid, nitric acid, sulfuric acid, toluenesulfonic acid, phosphoric acid, perchloric acid, or combinations thereof.

4. The process of claim 1 wherein the oxidation solvent further comprises an ionic liquid.

5. The process of claim 1 wherein the aromatic amide compound is hydrolyzed at a temperature of at least about 150° C.

6. The process of claim 1 wherein the oxidation product further comprises at least one additional amide compound and wherein the at least one additional amide compound is hydrolyzed to an aromatic carboxylic acid.

7. The process of claim 1 further comprising crystallizing at least a portion of the aromatic carboxylic acid in a crystallization zone to produce a solid product comprising a crystallized aromatic carboxylic acid.

8. The process of claim 7 wherein the oxidation solvent further comprises an ionic liquid and wherein crystallizing at least the portion of the aromatic carboxylic acid comprises adding a crystallization solvent, the crystallization solvent having a first composition, wherein the crystallization solvent comprises water, an acid, a hydroxide capable of hydrolyzing the aromatic amide compound, or combinations thereof, and wherein the aromatic carboxylic acid is less soluble in the crystallization solvent than in the oxidation solvent.

9. The process of claim 8 wherein the crystallization solvent crystallizes at least the portion of the aromatic carboxylic acid and hydrolyzes the aromatic amide compound.

10. The process of claim 8 wherein the crystallization solvent is the same as the hydrolyzing agent.

11. The process of claim 8 further comprising changing a composition of the crystallization solvent from the first composition to a second composition, wherein the aromatic carboxylic acid is less soluble in the second composition than in the first composition.

12. The process of claim 7 further comprising separating the solid product from a liquid portion.

13. The process of claim 7 wherein the aromatic carboxylic acid is crystallized before the aromatic amide compound is hydrolyzed, and wherein the solid product further comprises the aromatic amide compound, the process further comprising:
separating the solid product from a first liquid portion before the aromatic amide compound is hydrolyzed; and
hydrolyzing the aromatic amide compound in the solid product.

14. The process of claim 13 wherein the aromatic amide compound is hydrolyzed at a temperature of at least about 150° C. and further comprising:
separating the solid product after hydrolysis from a second liquid portion.

15. The process of claim 14 further comprising:
reducing the temperature of the separated solid product; and
washing the separated solid product with a washing solvent.

16. The process of claim 15 wherein the temperature of the washing solvent is less than about 125° C., and wherein the washing solvent is a second acid, or water, or both.

17. The process of claim 1 further comprising recycling at least a portion of at least one of the oxidation solvent, the ammonia or ammonium compound, the bromine source, the catalyst, the oxidizing agent, and the hydrolyzing agent to the reaction zone.

18. The process of claim 1 in which an amount of aromatic amide compound after hydrolysis is less than about 7% of an amount of aromatic amide compound by mass in the oxidation product.

19. The process in claim 1 in which a nitrogen content in the aromatic carboxylic acid after hydrolysis is less than about 2000 ppm by mass.

20. The process of claim 1 in which a nitrogen content in the aromatic carboxylic acid after hydrolysis is less than about 5% of an amount of nitrogen by mass in the oxidation product.
